(19) United States
(12) Patent Application Publication
(10) Pub. No.: US 2010/0292209 A1
(43) Pub. Date: Nov. 18, 2010

(54) METHODS FOR IDENTIFYING COMPOUNDS THAT MODULATE NEUROTROPHIC FACTOR SIGNALING

(75) Inventors: Letian Kuai, Waltham, MA (US); Stephen J. Haggarty, Dorchester, MA (US)

Correspondence Address: WOLF GREENFIELD & SACKS, P.C., 600 ATLANTIC AVENUE, BOSTON, MA, 02210-2206, US

(73) Assignees: Massachusetts Institute of Technology, Cambridge, MA (US); The General Hospital Corporation d/b/a Massachuset ts General Hospital, Boston, MA (US)

(21) Appl. No.: 12/594,049

(22) PCT Filed: Mar. 28, 2008
(86) PCT No.: PCT/US08/04107

§ 371 Date: May 18, 2010

Related U.S. Application Data
(60) Provisional application No. 60/921,219, filed on Mar. 30, 2007.

(51) Int. Cl. A61K 31/517; A61K 31/5377; A61K 31/407; A61K 31/553; A61P 25/00; A61P 25/28; A61P 25/18; A61P 25/24

(52) U.S. Cl. 514/211.09; 514/266.4; 514/234.5; 514/266.3; 514/410

(57) ABSTRACT

The invention relates to screening for compounds and compositions that modulate neurotrophic factor signaling as reflected by modulation of factor-induced neurite outgrowth. The compounds and compositions are useful in the treatment of a variety of disorders. The invention also provides methods for preparing compounds for treatment of such disorders.

### RELATED APPLICATION

This application claims the benefit under 35 U.S.C. §119(e) of U.S. provisional application Ser. No. 60/921,219 filed on Mar. 30, 2007, the entire disclosure of which is incorporated herein by reference.

### FIELD OF THE INVENTION

The invention relates to screening for compounds and compositions that modulate neurotrophic factor signaling as reflected by modulation of factor-induced neurite outgrowth. The compounds and compositions are useful in the treatment of a variety of disorders. The invention also provides methods for preparing compounds for treatment of such disorders.

### BACKGROUND OF THE INVENTION

The genes encoding Neuregulin1 (Nrg1) and its receptor, ErbB4, have been suggested to be risk genes for schizophrenia by a number of genetic association studies.¹⁻⁴ Nrg1 is believed to activate ErbB receptors in either paracrine or juxtacrine signaling mode depending on the splicing subtype of the erbB4 receptor.⁵ Some biological evidence also suggests a relationship between Nrg1-ErbB4 signaling and schizophrenia. Some experimental data supports underfunctioning of this pathway and other evidence supports possible over activity of this pathway. However, neither the genetics nor the biology has yet provided an unambiguous connection between this signaling system and the risk of schizophrenia.

Chemical genetics is an important approach to decipher the molecular circuitry that regulates biological phenotypes. This approach can exploit high-throughput (HTS) phenotypic assays to identify compounds that perturb a biological system followed by identification of their macromolecular targets.⁶ Although chemical genetic screening has been widely used in various biological systems, the approach has not been widely used to find new approaches to understand or treat psychiatric illness.⁷

### SUMMARY OF THE INVENTION

Here we establish a simple HTS system based on live cell-imaging to study neuronal differentiation induced by Nrg1 via the ErbB4 receptor and identify novel compounds that modulate the effect. The use of such compounds for treating psychotic or cognitive disorders, particularly schizophrenia, also is provided.

More broadly, the invention provides methods of screening for compounds and compositions that modulate neurotrophic factor signaling, as reflected by modulation of factor-induced neurite outgrowth. The compounds and compositions are useful in the treatment of a variety of disorders. The invention also provides methods for preparing compounds for treatment of such disorders.

According to one aspect of the invention, methods for identifying compounds or compositions useful as pharmacological agents for the treatment of psychotic or cognitive to disorders are provided. The methods include contacting a cell capable of neurite outgrowth, which cell expresses ErbB4 and optionally a fluorescent protein, with neuregulin-1 (Nrg1) and a compound or composition, and determining the neurite outgrowth of the cell. Modulation of neurite outgrowth relative to a control amount of neurite outgrowth is an indication that the compound or composition is a candidate pharmacological agent is useful in the treatment of a psychotic or cognitive disorder.

According to a second aspect of the invention, methods for identifying compounds or compositions that modulate Nrg1-ErbB4 signaling are provided. The methods include contacting a cell capable of neurite outgrowth, which cell expresses ErbB4 and optionally a fluorescent protein, with neuregulin-1 (Nrg1) and a compound or composition, and determining the neurite outgrowth of the cell, wherein modulation of neurite outgrowth relative to a control amount of neurite outgrowth is an indication that the compound or composition modulates Nrg1-ErbB4 signaling.

In some embodiments, the methods further include determining a second amount of neurite outgrowth of the cell in the absence of the compound or composition, and using the second amount of neurite outgrowth as the control amount of neurite outgrowth.

In other embodiments, the methods further include determining the effect of the compound or composition on nerve growth factor (NGF)-induced neurite outgrowth by contacting the cell with NGF and the compound or composition and determining the neurite outgrowth of the cell.

In any of these methods, the neurite outgrowth preferably is determined by cell imaging, more preferably live-cell fluorescence imaging. In certain of these embodiments, the live-cell fluorescence imaging is performed by automated microscopy.

Any of these methods also can include screening the compound or composition by determining the effect of the compound or composition on phosphorylation of extracellular signal-regulated kinase (ERK) polypeptides. In such embodiments, the ERK phosphorylation preferably is measured by a phospho-specific antibody or an antigen-binding fragment thereof. In other of these embodiments, the ERK polypeptides preferably are contained within a cell, and the cell is contacted with the compound or composition. More preferably, the cell expresses ErbB4 and is contacted with neuregulin-1 (Nrg1) prior to determining ERK phosphorylation.

For the foregoing methods, the cell preferably is a neuron, glia, or neuronal cell, more preferably a PC12 cell, a SH-SY5Y cell or a Neuro2a cell.

Preferred psychotic or cognitive disorders include a brief psychotic disorder, a delusional disorder, a schizoaffective disorder, schizophrenia, a schizophreniform disorder, a substance-induced psychotic disorder, a psychotic disorder due to a medical condition, paraphrenia, bipolar disorder, psychosis associated with Parkinson's disease, Huntington's disease, manic-depressive psychosis, major depressive disorder with psychotic features, or a shared psychotic disorder. Preferably the psychotic or cognitive disorder is schizophrenia, which may be catatonic schizophrenia, disorganized schizophrenia or paranoid schizophrenia.

According to another aspect of the invention, cell lines comprising a neuron, glia, or neuronal cell modified to express ErbB4, preferably a JM-a Cyt-2 isoform, are provided. In some embodiments, the neuron, glia, or neuronal cell is transfected with an expression vector that encodes ErbB4. In other embodiments, the neuron, glia, or neuronal cell further comprises a fluorescent protein; preferably the neuron, glia, or neuronal cell expresses a fluorescent protein. In some preferred the neuron, glia, or neuronal cell is transfected with an expression vector that encodes the fluorescent protein.

A preferred fluorescent protein is a green fluorescent protein.

Preferred neuron, glia, or neuronal cells include a PC12 cell, a SH-SY5Y cell or a Neuro2a cell.

According to another aspect of the invention, cultures of the foregoing cell lines and cell populations of the foregoing cell lines are provided.

In yet another aspect of the invention, methods for treating a subject having or suspected of having a psychotic or cognitive disorder are provided. The methods include administering to a subject in need of such treatment an effective amount of an aminoquinazoline compound or a indolocarbazole compound as a treatment for the psychotic or cognitive disorder. Preferred compounds inhibit the effect of NGF on neurite outgrowth and/or potentiate the effect of Nrg1 on neurite outgrowth and/or increase the level of ErbB4 and/or increase the uptake of Nrg1 by cells.

Preferred aminoquinazoline compounds include gefitinib (Iressa), erlotinib (Tarceva), salts thereof, and solvates thereof. Preferred indolocarbazole compounds include indolo[2,3-a]carbazole compounds, salts thereof, and solvates thereof. Preferably the indolo[2,3-a]carbazole is furanosylated. More preferably, the furanosylated indolo[2,3-a]carbazole is K252a, analogs thereof, derivatives thereof, salts thereof, or solvates thereof. Preferred analogs or derivatives of K252a do not have a methyl group at the C2′ position, e.g., K252a-2 (K252a-Me).

In preferred methods, the psychotic or cognitive disorder is a brief psychotic disorder, a delusional disorder, a schizoaffective disorder, schizophrenia, a schizophreniform disorder, a substance-induced psychotic disorder, a psychotic disorder due to a medical condition, paraphrenia, bipolar disorder, psychosis associated with Parkinson's disease, Huntington's disease, manic-depressive psychosis, major depressive disorder with psychotic features, or a shared psychotic disorder. Preferably the psychotic or cognitive disorder is schizophrenia, which can be catatonic schizophrenia, disorganized schizophrenia or paranoid schizophrenia.

In preferred embodiments, the subject is a human.

In a further aspect of the invention, methods for preparing a drug for the treatment of a psychotic or cognitive disorder are provided. The methods include identifying a compound or composition that modulates Ngr1-induced neurite outgrowth and/or that modulates ErbB4-Nrg1 signaling, particularly using the methods described herein, and formulating the compound or composition for administration to a subject in need of such treatment.

In another aspect, the invention provides for use of the foregoing agents, compounds and molecules in the preparation of medicaments, particularly medicaments for the treatment of psychotic or cognitive disorders, preferably schizophrenia.

These and other aspects of the invention are described further below.

### BRIEF DESCRIPTION OF THE DRAWINGS

FIG. 1 Nrg1 induces neurite outgrowth and Erk1/2 phosphorylation in PC12-ErbB4-GFP cell

A. PC12-GFP (Vector) and PC12-ErbB4-GFP (ErbB4) cells were plated in a 96-well microplate at 1200 cells/well and incubated at 5% CO₂ 37° C. for 12 hours and then treated with 20 ng/ml Nrg1, 20 ng/ml NGF or untreated, as indicated, for 3 days. Transmitted light cell images were compared. B. Green fluorescent image of Nrg1-treated PC12-ErbB4-GFP cell was overlaid with transmitted light image. C. PC12-GFP (Vector) and PC12-ErbB4-GFP (ErbB4) were treated with 20 ng/ml Nrg1 or NGF for the indicated times. Cell extracts were subjected to immunoblotting with antibodies against ErbB4 or phosphor-p44/42 MAPK, respectively.

FIG. 2 Nrg1 and NGF induced neurite outgrowth is dose-dependent and quantitatively measurable

Cells were plated in 384-well assay plate at 400 cells/well for 12 hours and then untreated or treated with Nrg1 and/or NGF at concentrations as indicated. Green fluorescent image of cells were acquired automatically every 24 hours and analyzed. A. Demonstration of a typical neurite detection result. Upper panel: cell image; Lower panel: computer-generated mask of cell bodies and neurites. Average neurite outgrowth per cell was determined after neurite detection. B. Growth curve of neurites induced by Nrg1 and NGF at 20 ng/ml over a 4-day course. C. Dose response curve of Nrg1 and NGF determined at Day 2. D. Dose response curve of Nrg1 and NGF determined at Day 4. E. Dose response curve of Nrg1/NGF co-treatment determined at Day 2. F. Dose response curve of Nrg1/NGF co-treatment determined at Day 4.

FIG. 3 Automated screening against 400 kinase inhibitors

A. A work flow chart of high-throughput screening. Cells were plated in 384-well assay plate at 400 cells/well and incubated for 12 hours. Compounds were then pin transferred into the wells 30 mins before hormone treatment. Nrg1 and NGF were then introduced by automated-liquid dispensing to a final concentration of 20 ng/ml for each well. Fluorescent images of each well were acquired and analyzed after 48 hours incubation. B. A small scale screening against 400 kinase inhibitors. The effect of each inhibitor (shaded circles) against Nrg1 and NGF were directly compared by the mean neurite outgrowth per cell. The 752 DMSO wells were considered as 752 identities and presented in open circles. Three compounds that lead to further characterization in this study were marked by arrows. The compounds were categorized according to their relative activity compared to the majority of DMSO, gated by blue dashed lines. C. Among the 400 kinase inhibitors, 51 caused significant reduction of cell numbers and were considered cyto-toxic. The remaining 349 compounds were categorized into 9 categories according to their effect on NGF and Nrg1. The numbers of compounds in each category are listed. D. This figure shows additional chemical structures of the tested kinase inhibitors superimposed on the results also shown in FIG. 3B.

FIG. 4 Quinazoline derivatives specifically inhibit Nrg1-induced neurite outgrowth

A. Structures of AG1478 analogues. B. Cells were pretreated with 15 μM AG1478 for 30 minutes followed by 20 ng/ml Nrg1 or NGF. Average outgrowth per cell was measured after 2 days incubation. C. Cells were pretreated with Iressa or Tarceva at various concentrations for 30 minutes followed by 20 ng/ml Nrg1. Mean outgrowth per cell was measured after 2 days incubation. D. Cells were pretreated with 2 μM Iressa or DMSO for 30 minutes followed by 20 ng/ml Nrg1 or NGF and lysed after 5 minutes. Cell lysates were immunoprecipitated by anti-ErbB4 antibody followed by western blotting with antibodies against phospho-tyrosine and ErbB4. Phosphor-ERK1/2 and total Erk2 levels were determined by direct western blotting with cell lysates.

FIG. 5. Indolocarbazole derivatives potentiate Nrg1-induced neurite outgrowth

A. Cells were untreated or treated with 50 nM K252a for 30 minutes followed by 20 ng/ml Nrg1 and NGF. Pictures were taken after 2 days. B. Structures of representative K252a analogues, K252a-2, K252a-5 and K252a-8. For a complete list of K252s analogues, see supplement data. C. Cells were treated with DMSO, 50 nM K252a or its analogues for 30 minutes followed by treatment of 20 ng/ml Nrg1 or NGF. The mean outgrowth per cell was measured after 2 days incubation. D. Cells were pretreated with K252a or K252a-5 at various concentrations for 30 minutes followed by 20 ng/ml Nrg. Mean outgrowth per cell was measured after 2 days incubation.

FIG. 6. Iressa-conjugated agarose affinity-captures

A. Scheme of chemical modification and conjugation of Iressa. B. Cells (400/well on 384-well plate) were treated with Iressa, Iressa_{—}1 and Iressa_{—}2 at various concentration as indicated for 30 minutes followed by treatment with 20 ng/mL of Nrg1. The mean neurite outgrowth per cell was determined after 2 days incubation. C. PC12-ErbB4-GFP cells were lysed with modified RIPA buffer at 4° C. for 10 min and the cell lysate was cleared by centrifugation. The cleared lysate (0.40 ml) was tumbled with Iressa (10 μM) or DMSO (1:1000 v/v) as indicated at 4° C. for 30 min before addition of the iTrap resin (10 μA). The resulting mixture was tumbled at 4° C. for 12 hours. The suspension was centrifuged and the supernatant was discarded. The resin was washed with the above modified RIPA buffer (1.0 ml) for four times. The captured proteins were separated by SDS-PAGE. The Western immunoblotting experiment was then performed using anti-ErbB4 antibody.

FIG. 7. K252a increases the level of ErbB4 and uptake of Nrg1

A. Cells were treated without or with K252a (50 nM), K252a-Me (50 nM) for 30 min followed by treatment with or without 20 ng/mL Nrg1. Cells were lysed 12 hrs after Nrg1 treatment and analyzed by Western blot with anti-ErbB4 antibody. The bands that correspond to ErbB4 are indicated. B. Cells were treated without or with K252a (50 nM), K252a-Me (50 nM) for 12 hrs then fixed and immunostained with anti-ErbB4 and Alexa594 conjugated secondary antibody. GFP and Alexa594 fluorescent cell images were taken using a 20× objective. C. Cells were treated without or with K252a (50 nM) for 12 hrs followed by treatment of 20 ng/mL Alexa594-Nrg1 for 30 mins and fixed. GFP and Alexa594 fluorescent cell images were taken using a 40× objective.

### DETAILED DESCRIPTION OF THE INVENTION

The genes encoding neuregulin1 (Nrg1) and its receptor, ErbB4, have been suggested to be risk genes for schizophrenia by a number of reports. Biological evidence consolidating the relationship between Nrg1-ErbB4 signaling and schizophrenia is also suggestive. Neither the genetics nor the biology is certain. Finding selective modulators of Nrg1-ErbB4 signaling pathway may be an important approach to further elucidate the relationship of this signaling system to the human disease.

We have generated a PC12 cell line which co-expresses the ErbB4 receptor and green fluorescent protein (GFP). The cell line, allows us to quantify neurite outgrowth in a live cell imaging assay in which measurements are made on the entire population of cells in wells of a 384-well plate using automated microscopy. We have shown that we can quantify neurite outgrowth as a function of Nrg1 concentration down to low nanogram/ml levels.

This cell model provides an opportunity to characterize the neurotrophic effects of Nrg1-ErbB4 signaling pathway and compare these effects to those of the NGF-TrkA signaling pathway, which already exist and are functional in the PC12 system. Although Nrg1 and NGF both stimulate the differentiation of PC12-ErbB4-GFP cells, NGF's effect is slower than Nrg1 especially in the first two days after treatment. In addition, co-treatment of NGF dramatically increases final length of neurites in the presence of saturating amounts of Nrg1. Nrg1 treated cells also exhibits a distinct phosphor-tyrosine signature from cells treated with NGF. Therefore, it is possible to begin to define the signaling pathways that each protein uses to produce its biological effects.

We have used this screening system to screen for small molecules that would selectively inhibit or potentiate the effects of Nrg1 or NGF. We have identified several classes of compounds that can specifically affect the Nrg1-ErbB4 signaling pathway. These compounds will be extremely useful in studying the biology of Nrg1 and in treatment of schizophrenia and other psychotic or cognitive disorders.

The invention provides various methods for identifying compounds or compositions that are useful as pharmacological agents for the treatment of psychotic or cognitive disorders. The methods provided by the invention also are useful for identifying compounds or compositions that modulate neurotrophic factor signaling, as demonstrated specifically for Nrg1-ErbB4 signaling herein. Similar assays as described herein can be performed using cells and cell lines capable of neurite outgrowth in which one or more receptors for the neurotrophic factor of interest are expressed in the cells or cell lines. Additional neurotrophic factor signaling pathways that can be tested in the assays of the invention include: other neuregulin and ErbB receptors, Nerve growth factor (NGF) and TrkA receptor and p75 receptors; Brain-derived neurotrophic factor and TrkB receptors; Neurotrophin-3 (NT-3) and TrkC receptors; and Glial cell line-derived neurotrophic factors (GDNF) and Met receptors. Such assays can be used to identify compounds and compositions that modulate (e.g., increase or reduce) neurotrophic factor signaling. Based on the use of the assay described herein for identifying compounds or compositions that are useful as pharmacological agents for the treatment of psychotic or cognitive disorders, assays that examine signaling of neurotrophic factors other than Ngr1 will be useful for treatment of other disorders, such as neurodegenerative disorders (e.g., Huntington's disease, Alzheimer's disease, multiple sclerosis), inflammation and neuropathic pain.

The methods utilize cells that are capable of neurite outgrowth, such as neural, glia, and neuronal cells. The cells preferably are modified to express, are contact with or contain molecules that permit analysis of neurite outgrowth. Such molecules provide contrast with the surrounding environment and facilitate imaging. In preferred embodiments, the cells express one or more fluorescent proteins, such as green fluorescent protein, such that neurites are readily imaged with fluorescent detection equipment. Other examples of fluorescent proteins include cyan fluorescent protein and yellow fluorescent protein. The fluorescent proteins are found in various species such as jellyfish and can be selected for appropriate excitation and emission peaks, e.g., cyan, green, yellow, orange, red, or far-red fluorescence emission.

It also is possible to treat the cells with exogenously added molecules, such as various dyes, that facilitate imaging of neurites. For example, dyes that bind the cell membrane and provide contrast for imaging neurite outgrowth can be added to cell media for binding to neurites and cell bodies. Prior to imaging, the cell media may be substituted with media that does not contain the dye, to increase contrast between the background and the cells and neurites. Dyes include dyes for living cells (i.e., vital dyes), such as 5-carboxy-fluorescein diacetate AM (Molecular Probes, Eugene, Oreg.). Cells also can be labeled using antibodies that bind to the cell surface and are detectably labeled to permit ready visualization, such as fluorescently labeled monoclonal antibodies, e.g., Cy3-conjugated monoclonal antibodies.

The cells used in the assays express ErbB4. For cells that do not endogenously express ErbB4, such as PC12 cells, the cells are modified to express ErbB4 recombinantly. For example, as described in the Examples below, PC12 cells can be transfected with an expression vectors that directs the expression of ErbB4 polypeptide. Preferably the JM-a Cyt-2 isoform of ErbB4 is used. Additional methods, vectors, etc. for recombinantly expressing polypeptides are well known in the art and may be adapted for expression of ErbB4.

A variety of cells are useful in the methods and assays of the invention. Preferred cells are neuron, glia or neuronal cells. Particular examples of the cells useful in the invention are PC12 cells, SH-SY5Y cells and Neuro2a cells.

For assaying the ability of compounds or compositions to modulate neurite outgrowth, the cells are contacted with neuregulin-1 (Nrg1), which induces a certain amount of neurite outgrowth. In certain embodiments the cells are contacted with nerve growth factor (NGF), either separate from or in combination with Nrg1. NGF also induces neurite outgrowth.

Appropriate negative controls typically are performed in parallel with the assays of the test compounds or compositions. Controls include: not contacting the cells with Nrg1 (and/or NGF in assays that include NGF-induced neurite outgrowth), not contacting the cells with the test compound or composition, and using cells that do not express ErbB4 (and/or Trk for NGF-containing assays). The control assays in which an added component of the assay is omitted can be performed by substituting for the component (e.g., Nrg1, NGF and/or the test compound or compositions) the vehicle used for adding the component to the assay. In the Examples below, for example, control assays are performed by substituting DMSO for the test compound. For cell controls, as shown in the examples, cells expressing fluorescent protein alone can be substituted for cells expressing both ErbB4 and fluorescent protein (e.g., using PC12-ErbB4-GFP cell line and the control cell line PC12-GFP). Thus, the assay can include determining a second amount of neurite outgrowth of the cell under control conditions (such as in the absence of the compound or composition or others as described herein), and using the second amount of neurite outgrowth as the control amount of neurite outgrowth. Typically, a plurality of assay mixtures are run in parallel with different compound or composition concentrations to obtain a different response to the various concentrations. Typically, one of these concentrations serves as a negative control, i.e., at zero concentration of agent or at a concentration of agent below the limits of assay detection.

The assays include contacting the cell capable of neurite outgrowth, with neuregulin-1 (Nrg1) and a compound or composition, and optionally NGF. The Nrg1 (and/or NGF) induces neurite outgrowth, and the effect of the compound or composition on neurite outgrowth is determined. Modulation of neurite outgrowth relative to a control amount of neurite outgrowth is an indication that the compound or composition is a candidate pharmacological agent is useful in the treatment of psychotic or cognitive disorders. Modulation of neurite outgrowth relative to a control amount of neurite outgrowth also is an indication that the compound or composition is a modulator of ErbB4-Nrg1 signaling.

Various methods for measuring neurite outgrowth are known in the art. In preferred embodiments, the neurite outgrowth is determined by cell imaging, such as live-cell fluorescence imaging. One example of this is provided in the Examples below. In this particular preferred method, cells are grown in multiwell plates such as 96-well or 384-well plates, and imaging is performed using an automated microscope. Pixel maps are generated by the analysis software (e.g., MetaXpress™). Cell bodies are identified as pixel blocks, preferably with an area smaller than 120 μm² but greater than 25 μm². Neurites are identified as line objects, e.g., those longer than 10 μm, and connected to each cell body. The neurite outgrowth for each well can be quantified as mean neurite length per cell.

At a suitable time after addition of the assay components, the plate is moved, if necessary, so that assay wells are positioned for measurement of signal. Because a change in the signal may begin shortly after addition of test compounds, it is desirable to align the assay well with the signal detector as quickly as possible, with times of about two seconds or less being desirable. In preferred embodiments of the invention, where the apparatus is configured for detection through the bottom of the well(s) and compounds are added from above the well(s), readings may be taken substantially continuously, since the plate does not need to be moved for addition of reagent. The well and detector device should remain aligned for a predetermined period of time suitable to measure and record the change in signal.

The apparatus of the present invention is programmable to begin the steps of an assay sequence in a predetermined first well (or rows or columns of wells) and proceed sequentially down the columns and across the rows of the plate in a predetermined route through well number n. It is preferred that the data from replicate wells treated with the same compound are collected and recorded (e.g., stored in the memory of a computer) for calculation of signal.

To accomplish rapid compound addition and rapid reading of the response, the detector can be modified by fitting an automatic pipetter and developing a software program to accomplish precise computer control over both the detector and the automatic pipetter. By integrating the combination of the fluorescence detection device (e.g., a microscope outfitted with appropriate detector(s)) and the automatic pipetter and using a microcomputer to control the commands to the detector and automatic pipetter, the delay time between reagent addition and detector reading can be significantly reduced. Moreover, both greater reproducibility and higher signal-to-noise ratios can be achieved as compared to manual addition of reagent because the computer repeats the process precisely time after time. Moreover, this arrangement permits a plurality of assays to be conducted concurrently without operator intervention. Thus, with automatic delivery of reagent followed by multiple signal measurements, reliability of the assays as well as the number of assays that can be performed per day are advantageously increased.

The assays also can include screening the compounds or compositions by determining the effect of the compound or composition on phosphorylation of extracellular signal-regulated kinase (ERK) polypeptides. Various methods for analyzing and optionally quantifying ERK phosphorylation are well known in the art. One preferred method is to measure ERK phosphorylation using a phospho-specific antibody or an antigen-binding fragment thereof. Such phospho-specific antibodies are commercially available.

The screening for effect on ERK phosphorylation can be performed in a cell based assay as described above, or alternatively in a non-cell based assay. For the cell based assays, the ERK polypeptide is contained within a cell (e.g., expressed by the cell endogenously or exogenously, such as recombinantly), and the cell is contacted with the compound or composition. In one preferred method, the cell expresses ErbB4 and is contacted with neuregulin-1 (Nrg1) prior to determining ERK phosphorylation. For non-cell based assays, the effect of the compound or composition on ERK phosphorylation can be assessed by a variety of in vitro protein assays known in the art.

The assays also can include screening the compounds or compositions by determining the effect of the compound or composition on ErbB4 levels (e.g., nucleic acid levels, polypeptide levels). Various methods for analyzing and optionally quantifying ErbB4 levels are well known in the art. One preferred method to measure ErbB4 levels is by using an antibody or an antigen-binding fragment thereof to detect ErbB4 polypeptide. Such antibodies are commercially available. Examples of this method is described in Example 3 below, which shows Western blot and immunofluorescence assays.

The assays also can include screening the compounds or compositions by determining the effect of the compound or composition on Nrg1 uptake by cells. Various methods for analyzing and optionally quantifying Nrg1 uptake by cells are well known in the art. One preferred method to measure Nrg1 uptake is by detecting the uptake into cells of a detectably labeled compound. An example of this method is described in Example 3 below, which uses Alexa594-labeled Nrg1.

The methods are useful for identifying compounds and compositions for use in treating psychotic or cognitive disorders, or for providing lead compounds and testing modified compounds that are useful in treating psychotic or cognitive disorders. Psychotic and cognitive disorders include a brief psychotic disorder, a delusional disorder, a schizoaffective disorder, schizophrenia, a schizophreniform disorder, a substance-induced psychotic disorder, a psychotic disorder due to a medical condition, paraphrenia, bipolar disorder, psychosis associated with Parkinson's disease, Huntington's disease, manic-depressive psychosis, major depressive disorder with psychotic features, or a shared psychotic disorder. Schizophrenia can be catatonic schizophrenia, disorganized schizophrenia or paranoid schizophrenia.

The methods also provides cell lines that can be used in performing the methods described herein. The cell lines include neuron, glia, or neuronal cells modified to express ErbB4. Various isoforms of ErbB4 are known in the art; a preferred isoform is the JM-a Cyt-2 isoform. Preferred cells for producing the cell lines include PC12 cells, SH-SY5Y cells and Neuro2a cells.

As noted above, the cells can be modified to express ErbB4 using a variety of expression vectors and a variety of methods to introduce such expression vectors into the cells. Such vectors and methods are well known in the art. A common method is to clone a coding sequence for ErbB4 into an expression vector and to then introduce the vector into the cell, e.g., via transfection, electroporation and the like. The expression vector can be integrated into the cell's DNA for stable expression of ErbB4.

The cell line also can be modified to contain one or more fluorescent proteins, which typically is done by recombinantly expressing one or more genes encoding the fluorescent proteins. The methods described herein and known in the art for cloning and expressing ErbB4 also are applicable for expression of the fluorescent proteins. A preferred fluorescent protein is green fluorescent protein, but as will be appreciated by those skilled in the art, any suitable fluorescent protein that provides the necessary detectable signal to permit imaging of neurite outgrowth can be used.

The invention also provides culture and cell population of the cell lines described herein.

Based on the discoveries described in more detail elsewhere herein, the invention further provides methods for treating a subject having or suspected of having a psychotic or cognitive disorder. The methods include administering to a subject in need of such treatment an effective amount of an aminoquinazoline compound or a indolocarbazole compound as a treatment for the psychotic or cognitive disorder. Preferred subjects are human, but other subjects may be treated in a similar manner, such as for testing of the compounds in animal models of the psychotic or cognitive disorders.

Particular psychotic or cognitive disorders treatable in this manner include a brief psychotic disorder, a delusional disorder, a schizoaffective disorder, schizophrenia, a schizophreniform disorder, a substance-induced psychotic disorder, a psychotic disorder due to a medical condition, paraphrenia, bipolar disorder, psychosis associated with Parkinson's disease, Huntington's disease, manic-depressive psychosis, major depressive disorder with psychotic features, or a shared psychotic disorder. Specific schizophrenia disorders include catatonic schizophrenia, disorganized schizophrenia or paranoid schizophrenia.

Preferred aminoquinazoline compounds include gefitinib, erlotinib, salts thereof, and solvates thereof. Gefitinib (also known as Iressa and ZD1839) is N-(3-chloro-4-fluoro-phenyl)-7-methoxy-6-(3-morpholin-4-ylpropoxy)quinazolin-4-amine and has the chemical formula C₂₂H₂₄ClFN₄O₃. Its structure is shown in FIG. 4A. Erlotinib (also known as Tarceva and OSI-774) is N-(3-ethynylphenyl)-6,7-bis(2-methoxyethoxy)quinazolin-4-amine and has the chemical formula C₂₂H₂₃N₃O₄. Its structure is shown in FIG. 4A. In some preferred embodiments, the aminoquinazoline compounds do not include AG1478 or PD158780.

Preferred indolocarbazole compounds include indolo[2,3-a]carbazoles, salts thereof, and solvates thereof. More preferably, the indolo[2,3-a]carbazole is furanosylated. Examples of furanosylated indolo[2,3-a]carbazoles include K252a, analogs thereof, derivatives thereof, salts thereof, and solvates thereof K252a is methyl (5R,6R,8R)-6-hydroxy-5-methyl-13-oxo-5,6,7,8,14,15-hexahydro-13H-5,8-epoxy-4b,8a,14-triazadibenzo[b,h]cycloocta[1,2,3,4-jkl]cyclopenta[e]-as-indacene-6-carboxylate and has the chemical formula C₂₇H₂₁N₃O₅. Various derivatives of K252a are known in the art; see e.g., U.S. Pat. No. 6,472,385; Schneider et al., Organic Letters 2005. 7(9): 1695-1698; Nheu et al., Cancer Journal 2002. 8(4):328-336; Sanchez et al., Nat Prod Rep 2006. 23:1007-4105; KT5853; and KT5720. The structures of K252a and several derivative are shown in FIG. 5B; the structure of K252c is shown in FIG. 3B.

The invention also provides methods for preparing a drug for the treatment of a psychotic or cognitive disorder, or for modulating neurotrophic signaling, particularly modulates ErbB4-Nrg1 signaling. For this preferred embodiment, compounds or compositions that modulate Ngr1-induced neurite outgrowth are identified in accordance with the methods described herein, and then are formulated for administration to a subject in need of such treatment.

The candidate compounds and compositions can be derived from, for example, combinatorial peptide libraries, small molecule libraries, or natural product libraries. Candidate compounds and compositions encompass numerous chemical classes, although typically they are organic compounds. Preferably, the candidate pharmacological agents are small organic compounds, i.e., those having a molecular weight of more than 50 yet less than about 2500. Candidate compounds and compositions comprise functional chemical groups necessary for structural interactions with polypeptides (e.g., kinase sites), and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid (i.e., aptamer), the agent typically is a DNA or RNA molecule, although modified nucleic acids having non-natural bonds or subunits are also contemplated.

Candidate agents are obtained from a wide variety of sources including libraries of synthetic or natural compounds. For example, numerous methods are available and known to one of ordinary skill in the art for random and directed synthesis of a wide variety of organic compounds and biomolecules, including expression of randomized oligonucleotides, random or non-random peptide libraries, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

The invention also relates in part to methods of treating disorders neurotrophic factor signaling, particularly Ngr1-ErbB4 signaling, such as psychotic or cognitive disorders, particularly schizophrenia. An “effective amount” of a drug therapy is an amount of a compound or composition as described herein that alone, or together with further doses, produces the desired response, e.g. modulation of neurotrophic factor signaling, particularly Ngr1-ErbB4 signaling and/or amelioration of the psychotic or cognitive disorder.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition, or reversing the physiological effects of the disease.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the agent that modulates neurotrophic factor signaling (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of one or more compounds or compositions as described herein for producing the desired response in a unit of weight or volume suitable for administration to a patient.

The doses of compounds or compositions administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Various modes of administration will be known to one of ordinary skill in the art which effectively deliver the compounds or compositions to a desired tissue, cell or bodily fluid. Administration includes: topical, intravenous, oral, intracavity, intrathecal, intrasynovial, buccal, sublingual, intranasal, transdermal, intravitreal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of compounds or compositions will be known to one of ordinary skill in the art, in which the dose amount, schedule of administration, sites of administration, mode of administration (e.g., intra-organ) and the like vary from those presented herein.

Administration to mammals other than humans of compounds or compositions, e.g. for testing purposes or veterinary therapeutic purposes, is carried out under substantially the same conditions as described above. It will be understood by one of ordinary skill in the art that this invention is applicable to both human and animal diseases that can be treated by the compounds or compositions as described herein. Thus this invention is intended to be used in husbandry and veterinary medicine as well as in human therapeutics.

In general, a therapeutically effective amount of a compound or composition typically varies from about 0.01 ng/kg to about 1000 μg/kg, preferably from about 0.1 ng/kg to about 200 μg/kg and most preferably from about 0.2 ng/kg to about 20 μg/kg, in one or more dose administrations daily, for one or more days. Lesser or greater amounts may be found to be therapeutically effective and thus also are useful in accordance with the invention.

The pharmaceutical preparations of the invention may be administered alone or in conjunction with standard treatment(s) of the disorders described herein, e.g., psychotic or cognitive disorders. For example, treatment for schizophrenia with a pharmaceutical agent of the invention, may be undertaken in parallel with treatments for schizophrenia that are known and practiced in the art.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds or compositions described herein may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the compounds or compositions, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds.

The pharmaceutical compositions also may contain, optionally, suitable preservatives, such as: benzalkonium chloride; chlorobutanol; parabens; and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A long-term sustained release implant also may be used for administration of the pharmaceutical agent composition. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating conditions by placing the implant near portions of a subject affected by such activity, thereby effecting localized, high doses of the compounds of the invention.

### EXAMPLES

### Example 1

PC12 cell has been used as a differentiation model for decades because of its ability to differentiate upon treatment with neurotrophins such as Nerve Growth Factor (NGF). Although the PC12 cell does not express ErbB4 and does not differentiate when treated with Nrg1, PC12 cells expressing human ErbB4 can differentiate upon treatment of Nrg1⁸. Therefore, we prepared a stable PC12 cell line that co-expresses Green Fluorescent Protein (GFP) and human ErbB4 isoform JMa-Cyt2 (For recent studies regarding other ErbB4 isoforms see references⁹⁻¹¹). We, however, did not notice significantly different neurite-induction in PC12 cells among these isoforms (data not shown).

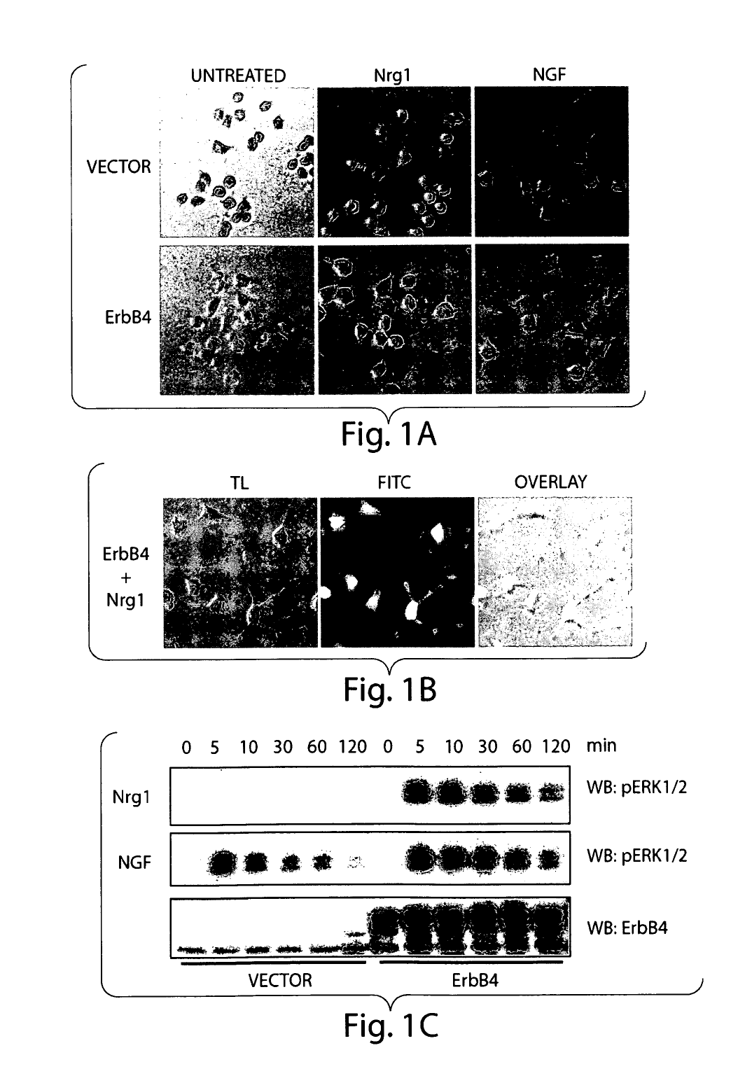

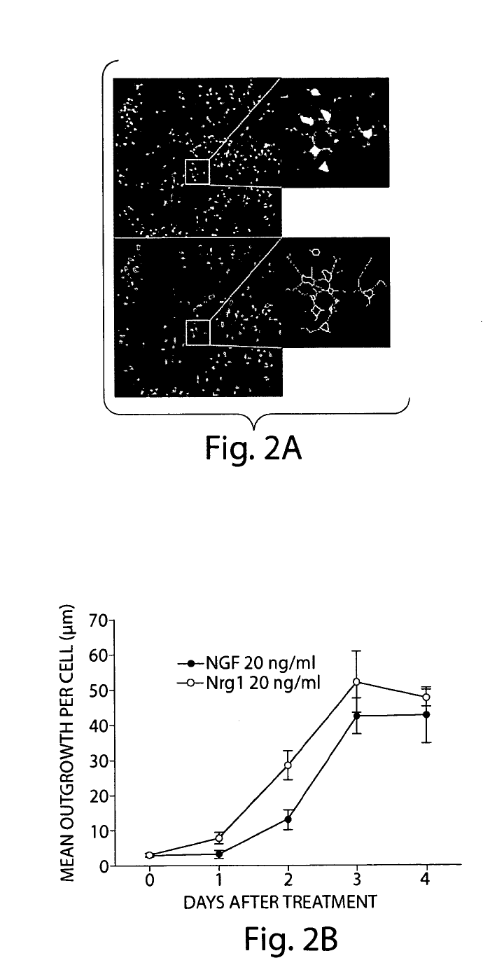

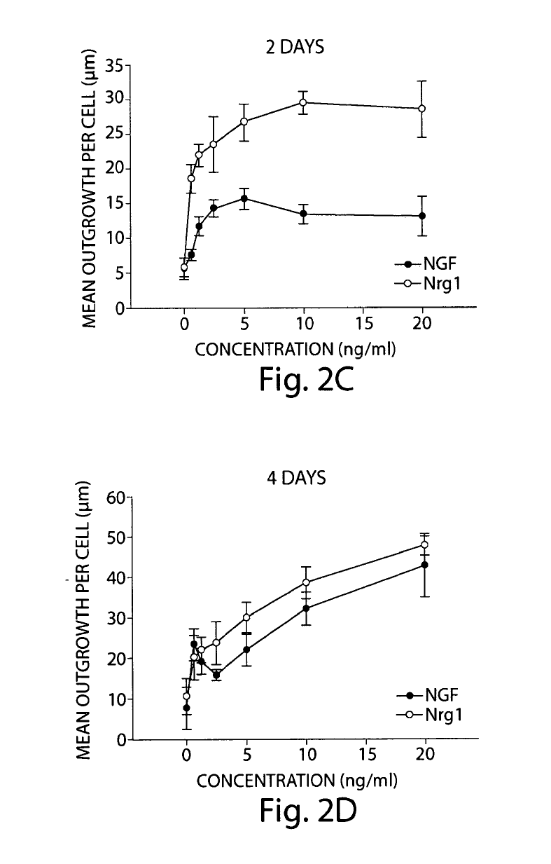

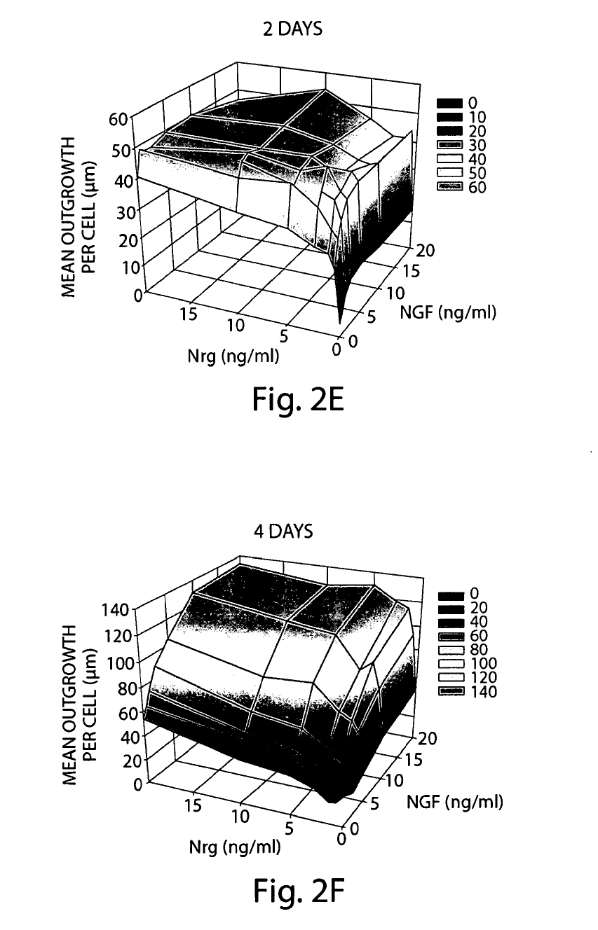

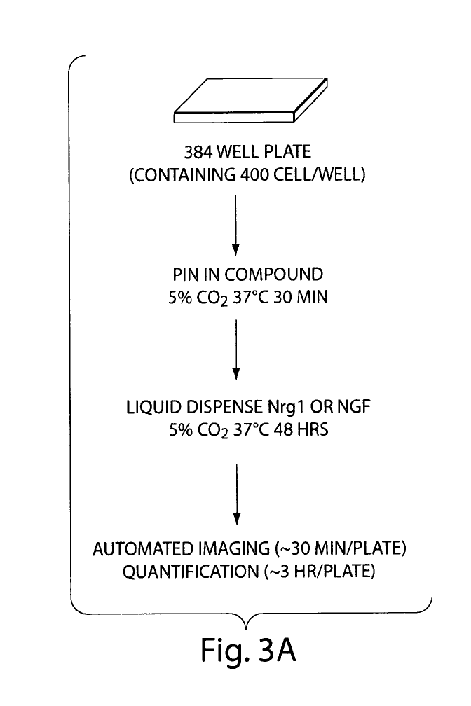

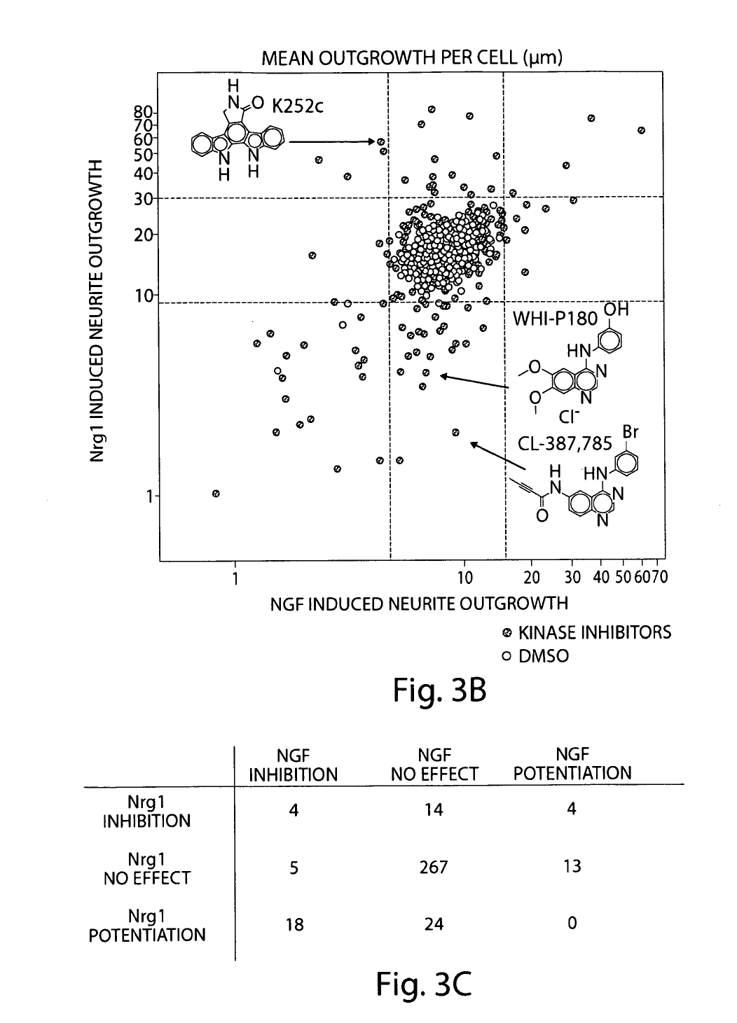

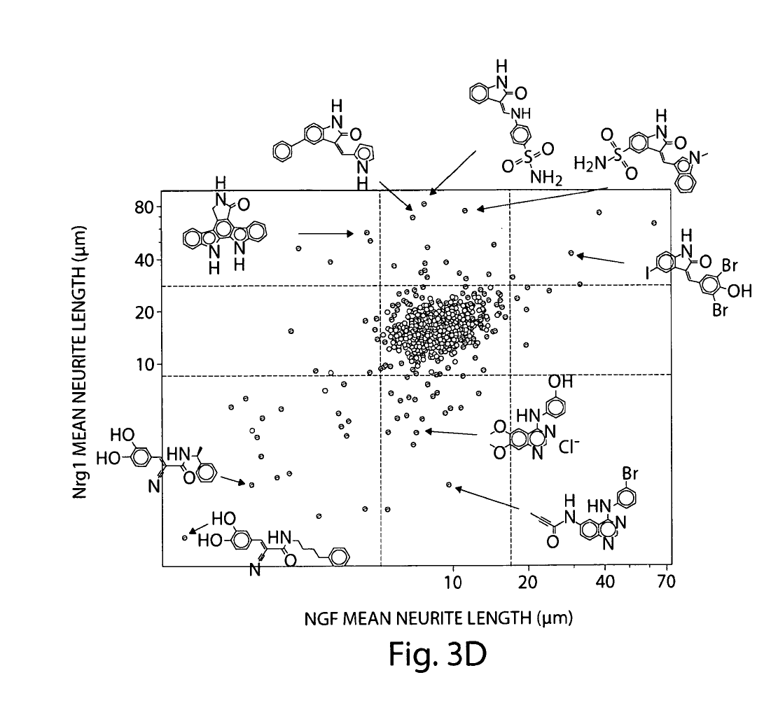

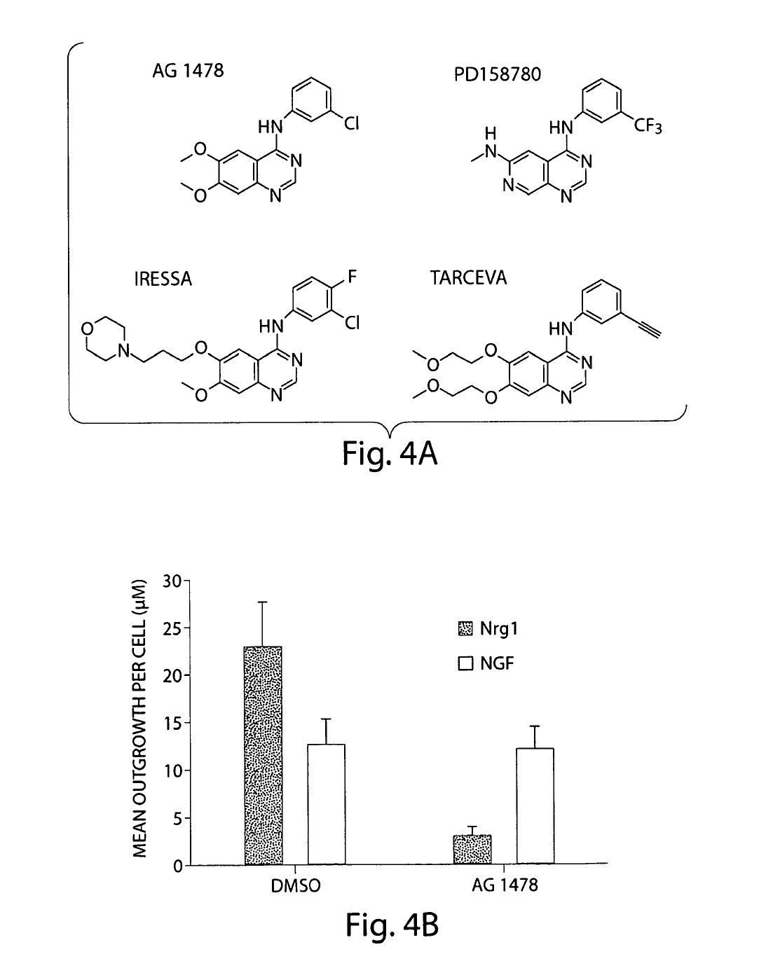

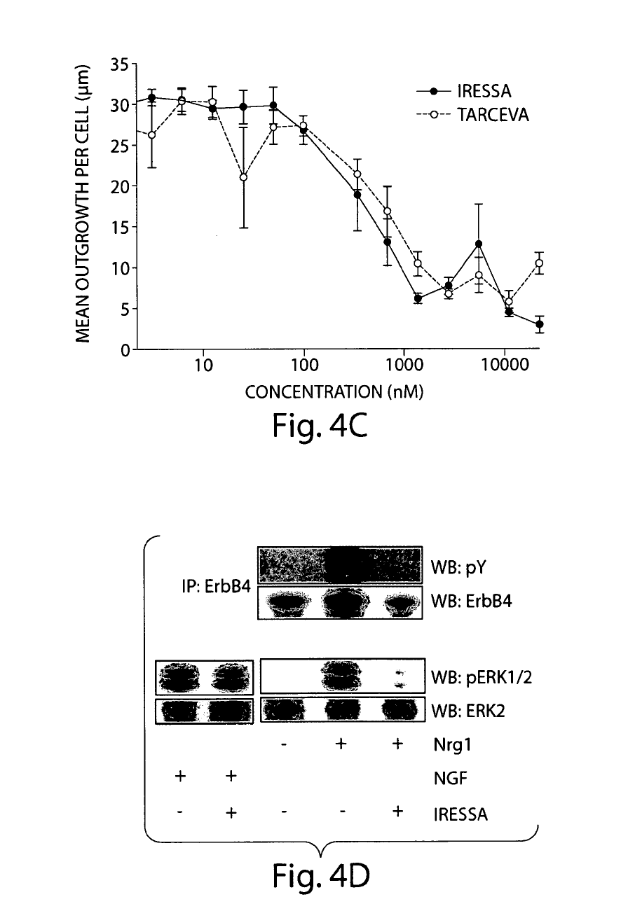

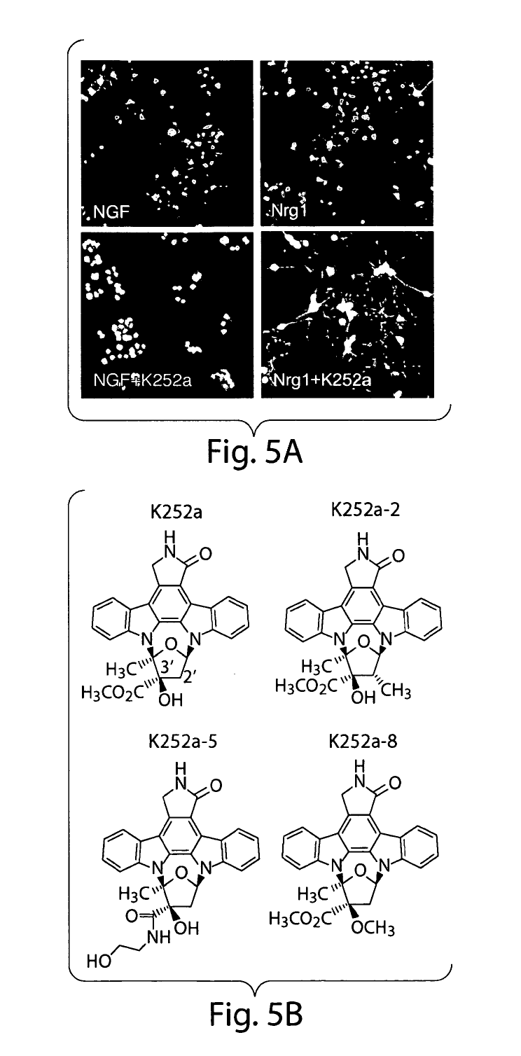

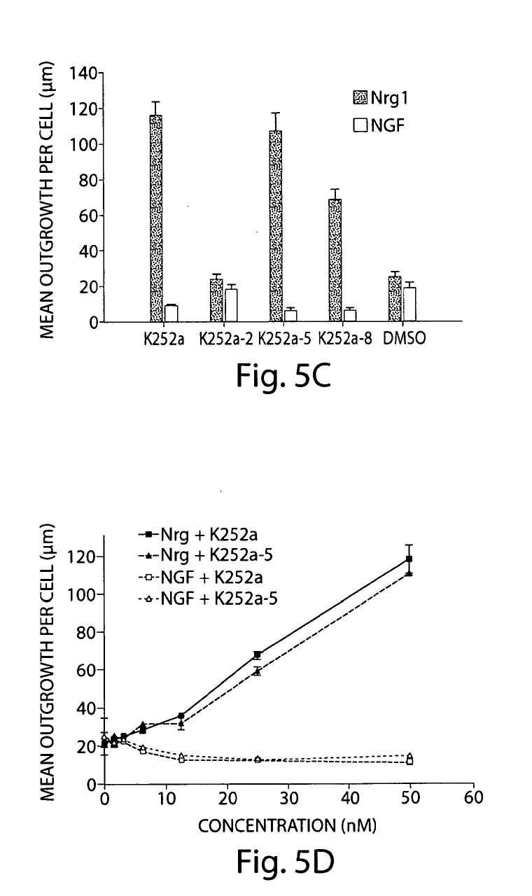

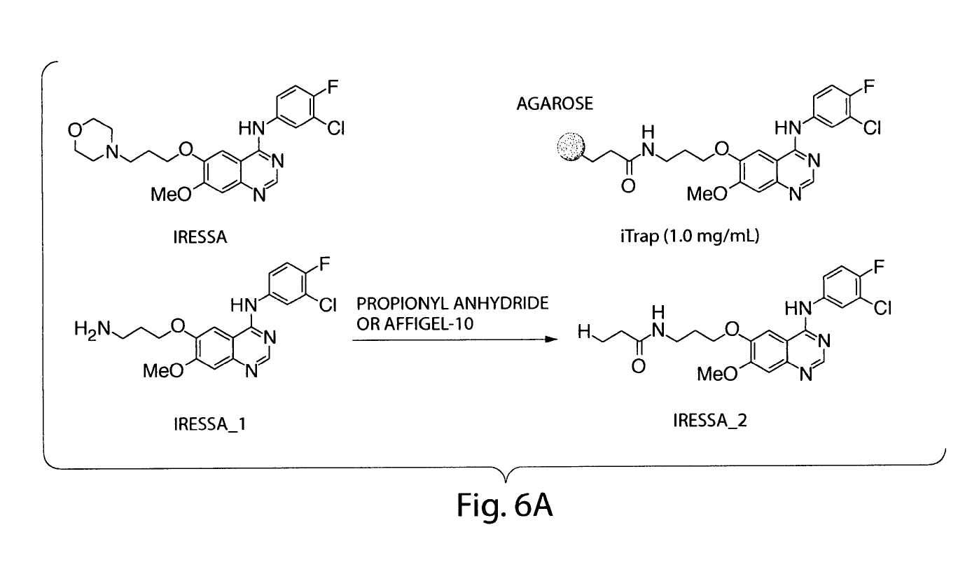

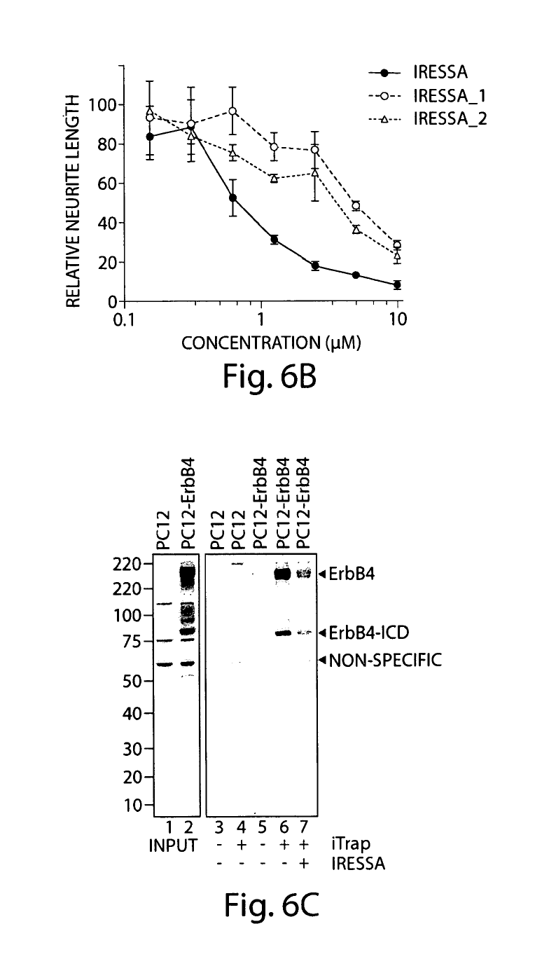

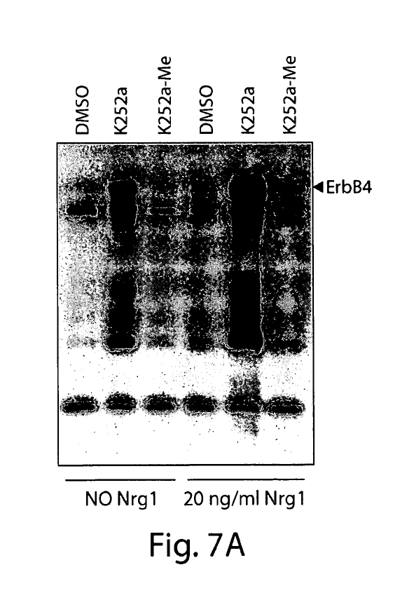

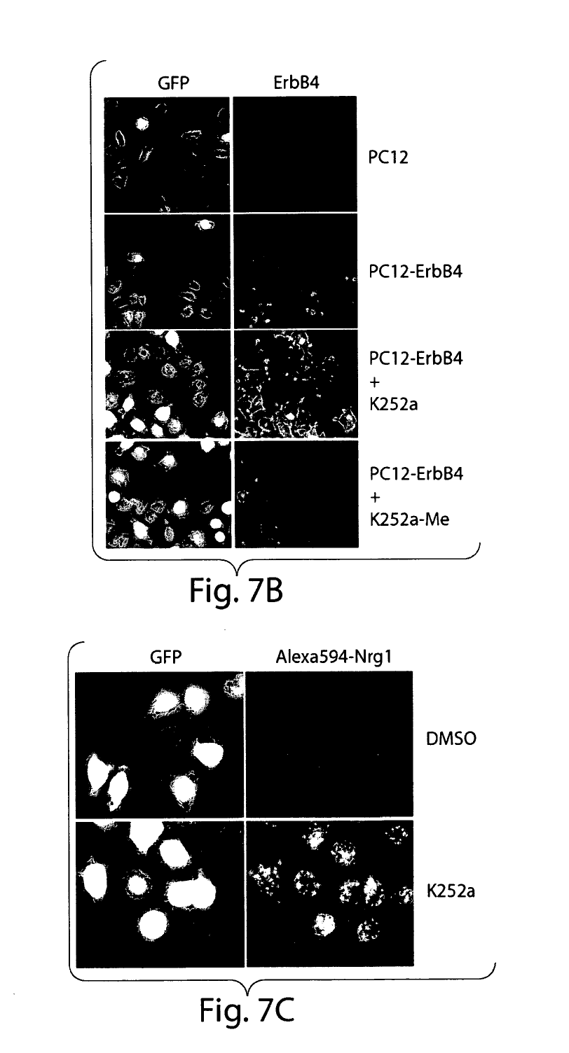

The PC12-ErbB4-GFP cell line and a control cell line, PC12-GFP, were examined for NGF or Nrg1-induced neurite outgrowth. Stimulation of PC12-ErbB4-GFP cells with Nrg1 for 3 days resulted in neurite outgrowth compared to PC12-GFP cells. Both PC12-ErbB4-GFP cells and PC12-GFP cells exhibit apparent differentiation when treated with NGF (FIG. 1A). The expressed GFP is localized throughout the cell body including the neurites. Under low magnification (<10×), the distribution of GFP is uniform and the fluorescent image reliably represents the whole cell body and the processes attached to it (FIG. 1B).

To verify the activity of the erbB4 signaling cascade, we examined ERK kinase phosphorylation in PC12-GFP cells and PC12-ErbB4-GFP cells. In both cell lines, NGF induces rapid phosphorylation of ERK/MAPK which peaked as early as 5 min after treatment. Nrg1 induces strong phosphorylation of ERK/MAPK only when ErbB4 is expressed (FIG. 1C).

Expression of soluble GFP in PC12 cell allows the use of automated microscopy to acquire live fluorescent image and analyze neurite outgrowth. PC12 cells can be cultured in tissue culture-treated 96 well or 384 well plates for up to 5 days without changing the medium. To minimize dumpiness of cell and intersection of neurites, we seeded cells at a low density of 4000 cell/cm². We found that Molecular Device ImageXpress 5000A automated microscope equipped with 4× objective is able to perfectly acquire one entire well of 384 well plates and the resulting image is sufficient to detect the neurites. A typical pixel map generated by the analysis software MetaXpress™ is demonstrated in FIG. 2A. Cell bodies were identified as pixel blocks with area smaller than 120 μm² but greater than 25 μm² and the neurites were subsequently identified as line objects longer than 10 μm and connected to each cell body. The neurite outgrowth for each well was therefore quantified as mean neurite length per cell.

To study the robustness of automated neurite detection, we examined the dose effect of Nrg1 and NGF on inducing neurite outgrowth. PC12-ErbB4-GFP cells were treated with Nrg1, NGF or both at various doses and images were acquired every 24 hours and quantified. Both NGF and Nrg1 stimulated a continuous increase of average neurite length over a 4-day course. NGF-treated cells appeared to differentiate slower than Nrg1-treated cells in the first 2 days but at day 4 the average lengths of neurite per cell were comparable (FIG. 2B). This might be explained by an up-regulation of TrkA¹² or a secondary receptor of NGF, P75NTR¹³, by TrkA activation. Significant cell detaching, dumpiness and decreased GFP signal were observed after longer incubation than 5 days and eventually caused false neurite detection. The average neurite length exhibits a strong correlation to the dose of NGF or Nrg1 administered especially under 10 ng/ml at day 2 (FIG. 2C) and under 20 ng/ml at day 4 (FIG. 2D), which might reflect a decrease of actual concentration of the growth factors in the medium. Although longer exposure is potentially achievable by changing medium, we conclude that current automated neurite detection with 4 day incubation can reliably report the effect of neurite induced by NGF and Nrg1 and is sufficient to study quantitatively the kinetics of neurite outgrowth. Interestingly, co-treatment of NGF and Nrg1 at least additively, if not synergistically, enhanced the average length of neurites at all the concentrations tested. Most importantly, NGF and Nrg1 can potentiate each other even at their saturating concentrations (FIG. 2E, 2F), indicating each receptor activation might not exhaust the differentiation capacity and the two signaling pathways merge at certain level and can be further potentiated.

Nrg1 and NGF activate receptor tyrosine kinase pathways and induce a cascade of kinase event which plays key role to the neurotrophic effect. Perturbation of kinases is expected to modulate Nrg1 and NGF signaling. To identify kinase inhibitors that can specifically modulate Nrg1 or NGF induced neurite outgrowth, we screened 400 known small molecule kinase inhibitors at single dose (10 micromolar). A total of 400 compounds, together with 752 DMSO controls, were pin-transferred into 384 well plates containing PC12-ErbB4-GFP cells prior to treatment of Nrg1 or NGF. Cell images were acquired after 48 hours and quantified (FIG. 3A). In such a system, NGF induced a mean neurite length of 8.7±1.5 μm in the presence of DMSO while Nrg1 induced a mean neurite length of 15.7±2.6 μm. The 752 DMSO controls exhibit a strong consistency and only 3 appeared to be abnormal (FIG. 3B). Within the 400 kinase inhibitors, 51 lead to significant cell number reduction in either Nrg1 or NGF (less than 100 cells after two day incubation) and were therefore considered cytotoxic. The remaining 349 compounds were categorized into 9 categories based on their relative activity compared to DMSO and specificity on Nrg1 and NGF induced neurite length (FIGS. 3B and C).

Single dose kinase inhibitor screening revealed two quinazoline derivative kinase inhibitors that inhibited Nrg1-induced neurite outgrowth but not NGF (FIG. 3B). Another commonly used quinazoline derivative, AG1478 [4-(3-Chloroanilino-6,7-dimethoxy)-quinazoline] (FIG. 4A), was known to competitively bind to the ATP pocket of EGFR (also known as ErbB1)¹⁴ and selectively inhibit ErbB1 over ErbB2¹⁵. Little is known about its ability of inhibiting ErbB4. Interestingly, we noticed that 1 μM AG1478 can specifically inhibit the Nrg1-induced neurite outgrowth but not NGF induced neurite outgrowth. (FIG. 4B). It has been reported that PD158780 (FIG. 4A), a very close analogue of AG1478, decreases Nrg1 induced neurite outgrowth in cultured hippocampal neurons¹⁶ and reverses the decrement in current caused by Nrg1 in PFC pyramidal neuron current assay¹⁷, suggesting that molecules with similar structure may indeed be possible modulators of Nrg1 signaling. We therefore further tested two other molecules that share same structural scaffold with AG1478, Iressa and Tarceva, which are also FDA approved drugs for non-small lung cancer based on their potent inhibition of EGFR. Both drugs dose-dependently inhibited Nrg1-induced neurite outgrowth with an IC₅₀ of 500 nM (FIG. 4C). NGF-induced neurite outgrowth was not inhibited in the concentration range of 100 nM˜1 (data not shown). Indeed, the phosphorylation of ErbB4 receptors induced by Nrg1 was inhibited by 1 μM Iressa and the subsequent phosphorylation of ERK/MAPK was also diminished. On the other hand, Iressa did not affect NGF-induced MAPK activation (FIG. 4D).

We also noticed that an indolocarbazole family kinase inhibitor, K252c, potentiates Nrg1 induced neurite outgrowth. The indolocarbazole family compounds, among which staurosporine and K252a have been investigated extensively in the past decades, were often viewed as broad-spectrum kinase inhibitors¹⁸. K252a was also known as a very potent TrkA inhibitor and was widely used for inhibition of NGF-induced processes^{19,20,21}. Consistently, in PC12-ErbB4-GFP cells, K252a completely inhibited NGF-induced neurite outgrowth at 10 nM. However, similar to K252c, K252a potentiated Nrg1-induced neurite outgrowth at same concentration range (FIG. 5A).

We further tested a series of 21 K252a derivatives that bear modification at various positions. Three representative molecules are demonstrated in FIG. 5B. Interestingly, the NGF-inhibiting and Nrg1-potentiating activities of K252a were both diminished in K252a-2 (FIG. 5C), which contains a single methyl modification at C2′ position²². In fact, three out of 21 tested analogues bear modification at the same position and they all lose the activity indicating that C2′-position may be critical for both NGF inhibition and Nrg1 potentiation. Certain substitution at C3′ position, such as K252a-5 and K252-8, did not or modestly affect the activity (FIG. 5C). In addition, K252a-5 appears to have similar potency as K252a (FIG. 5D). Indeed, K252a has been shown to have a neuroprotective effect in several cell types via Trk family receptor²³. Another K252a derivative, K252b, has much lower potency on inhibiting NGF-TrkA signaling but potentiates trophic action of neurotrophin-3, which facilitates TrkC receptor²⁴. However, the detailed mechanism for K252a-derivatives on neurite outgrowth is not known. The discovery that Nrg1 signaling can also be potentiated by K252a-derivatives indicates that ErbB receptor-signaling is susceptible to potentiation. It is even possible that K252a is a potent modulator for a common downstream component shared by all the neurotrophic factors except that in the NGF case it appears to be an inhibitor because it also blocks TrkA activation and the subsequent signal transduction. A thorough exploration of the structure activity relationships of this chemical structure will be necessary to understand these observations more fully.

Nrg1 has been suggested to be a risk gene for schizophrenia, although a specific sequence variant has not been identified. In the body of work on this suggestion, it is not clear whether gain, loss or change of function is related to the illness. To further elucidate the relationship between Nrg1-ErbB4 signaling and schizophrenia, both negative and positive perturbations will be important. In the present study we established a morphology-based high-throughput screening system to find modulators of Nrg1-ErbB4 signaling by quantitatively measuring neurite-induction. By screening a small collection of kinase inhibitors, two classes of compounds were identified to specifically potentiate or inhibit Nrg1-induced neurite outgrowth. Further investigation of the electrophysiological and biochemical effects of these compounds and their structure-activity relationship will provide more insight in understanding the role of Nrg1-ErbB4 signaling in neuronal biology and processes regulated by Nrg1. A larger library screening is in progress and we anticipate that more novel interesting compounds that specifically modulate Nrg1-ErbB4 will be identified.

### Materials and Methods

### Materials

PC12 cells (subclone Neuroscreen™-1) were obtained from Cellomics (now ThermoFisher Scientific, Pittsburgh, Pa.). pcDNA3-ErbB4 is a kind gift of Dr. S. R. Vincent, University of British Columbia, Canada. Antibodies used are: rabbit anti-ErbB4 c-18 (Santa Cruz Biotechnology, Santa Cruz, Calif.), rabbit anti-phospho-P42/44MAPK, rabbit anti-P42MAPK (Cell Signaling Technology), mouse anti-Phosphotyrosine 4G10 (Upstate, Charlottesville, Va.). Other tissue culture and molecular biology reagents are described in Methods.

### Cell Culturing

PC12 cells were maintained in RPMI 1640 media containing 10% heat inactivated horse serum, 5% heat inactivated fetal bovine serum and 1% penicillin/streptomycin. For PC12-ErbB4-GFP and PC12-GFP, 1% penicillin/streptomycin was replaced with 750 μg/ml Gentamicin (Gibco). Cells were passaged at 80-90% confluency and incubated at 37° C. in 5% CO₂. Media was changed every 3 days.

### Stable Cell Line Establishment

PC12 cells were co-transfected with pcDNA3-ErbB4-neomycin or pcDNA3-neomycin and pcDNA-GFP using FuGene 6 transfection reagent (Roche Diagnostics). Cells that express neomycin resistant gene were selected and maintained in same culture media with substitution of 750 μg/ml Gentamicin as selection agent and anti-biotic. After 2 weeks Gentamicin selection, cells were further selected by Fluorescent Activated Cell Sorting (FACS) for the top 5% population that strongly expresses GFP. The expression of GFP in the resulting cell populations, PC12-ErbB4-GFP and PC12-GFP, was observed to be stable for at least 50 passages.

### Immunoprecipitation and Western Blot

Cells were lysed with RIPA buffer (Pierce Technology, Rockford, Ill.) containing 1 tablet/10 ml protease inhibitor cocktail Complete Mini (Roche Applied Science). For phosphoprotein analysis, Halt Phosphatase Inhibitor Cocktail (Pierce Technology) was also included. Cell lysate was cleared by centrifuge at 15,000 rpm for 30 min followed by addition of LDS sample buffer (Invitrogen) for direct analysis or immunoprecipitated with specific primary antibodies and Protein A/G agarose (Pierce Technology) following the manufacturer's protocol. Samples were separated in 4-12% gradient SDS-PAGE and transferred to a polyvinylidene difluoride (PVDF) membrane in 25 mM Tris, 192 mM glycine and 20% methanol. The membrane was probed with specific primary antibodies according to specified recipes provided by their vendors and then horse radish peroxidase-conjugated secondary antibody to mouse or rabbit IgG (GE Healthcare, Piscataway, N.J.). Target protein bands were detected with SuperSignal West Femto Max Sensitivity Substrate (Pierce Technology).

### Cell Imaging

Cells were seeded in tissue culture-treated 96 well or 384 well plates at typical density of 8000 cell/cm². Even distribution was achieved by a quick centrifuge at 500 rpm shortly after seeding. Cells were then incubated for 12 hours followed by treatment of growth factor or chemical compounds. At specified time points, fluorescent images were taken under ImageXpress® 5000A or ImageXpress® Micro automated microscopy (Molecular Devices) either manually or automatically at 4× magnification or as specified. Transmitted light images were taken under ImageXpress Micro with the attached transmitted light device.

### Neurite Detection and Analysis

Neurite detection and analysis were performed with MetaXpress™ (Molecular Devices) using “Neurite Detection” analysis module. Cell bodies were specified as pixel blocks of minimum width 8 μm, maximum area 150 μm² and intensity 1000 above local background. Neurites were specified as linear objects with maximum width 3 μm and intensity 500 above local background.

### Example 2

### An Iressa-Conjugated Agorase Affinity-Captures ErbB4

Agarose beads (iTrap) conjugated with a derivative of Iressa (Iressa_{—}2, FIG. 6A) were used to examine the affinity of Iressa against ErbB4. To ensure that the chemical modification did not cause dramatic loss of activity, the activity of intermediates of the conjugation were verified by neurite outgrowth assay and appeared comparable to Iressa (FIG. 6B). The iTrap was able to precipitate ErbB4 from PC12-Erbb4 lysates compared to un-conjugated beads. More importantly, the precipitation was attenuated by 10 uM free Iressa, suggesting that Iressa indeed binds to ErbB4 and replaces iTrap.

### Example 3

### K252a Increases the Level of ErbB4 and Uptake of Nrg1

We noticed that the level of ErbB4 in PC12-ErbB4 was significantly increased after 12 hrs treatment of K252a but not K252a-Me (FIG. 7A). This phenomenon, confirmed by immunofluorescence staining for ErbB4 in PC12-ErbB4 (FIG. 7B), might explain why K252a potentiate Nrg1-induced neurite outgrowth. To verify that increased level of ErbB4 does contribute to neuregulin signaling, we examined the Nrg1 uptake with Alexa594-labeled Nrg1 (FIG. 7C). Cells were treated with K252a for 12 hrs followed by Alexa594-labeled Nrg1 for 30 min. The Nrg1 uptake is significantly higher in K252a-treated cells compared to DMSO-treated cells indicating a faster internalization, which is necessary for Nrg1 signaling (25).

### REFERENCES

1. Ross, C. A., Margolis, R. L., Reading, S. A., Pletnikov, M. & Coyle, J. T. Neurobiology of schizophrenia. Neuron 52, 139-53 (2006).
2. Harrison, P. J. & Law, A. J. Neuregulin 1 and schizophrenia: genetics, gene expression, and neurobiology. Biol Psychiatry 60, 132-40 (2006).
3. Silberberg, G., Darvasi, A., Pinkas-Kramarski, R. & Navon, R. The involvement of ErbB4 with schizophrenia: association and expression studies. Am J Med Genet B Neuropsychiatr Genet. 141, 142-8 (2006).
4. Hahn, C. G. et al. Altered neuregulin 1-erbB4 signaling contributes to NMDA receptor hypofunction in schizophrenia. Nat Med 12, 824-8 (2006).
5. Falls, D. L. Neuregulins and the neuromuscular system: 10 years of answers and questions. J Neurocytol 32, 619-47 (2003).
6. Stockwell, B. R. Exploring biology with small organic molecules. Nature 432, 846-54 (2004).
7. Smukste, I. & Stockwell, B. R. Advances in chemical genetics. Annu Rev Genomics Hum Genet. 6, 261-86 (2005).
8. Vaskovsky, A., Lupowitz, Z., Erlich, S. & Pinkas-Kramarski, R. ErbB-4 activation promotes neurite outgrowth in PC12 cells. J Neurochem 74, 979-87 (2000).
9. Carpenter, G. ErbB-4: mechanism of action and biology. Exp Cell Res 284, 66-77 (2003).
10. Elenius, K. et al. Characterization of a naturally occurring ErbB4 isoform that does not bind or activate phosphatidyl inositol 3-kinase. Oncogene 18, 2607-15 (1999).
11. Elenius, K. et al. A novel juxtamembrane domain isoform of HER4/ErbB4. Isoform-specific tissue distribution and differential processing in response to phorbol ester. J Biol Chem 272, 26761-8 (1997).
12. Vaghefi, H. & Neet, K. E. Deacetylation of p53 after nerve growth factor treatment in PC12 cells as a post-translational modification mechanism of neurotrophin-induced tumor suppressor activation. Oncogene 23, 8078-87 (2004).
13. Rankin, S. L., Guy, C. S. & Mearow, K. M. TrkA NGF receptor plays a role in the modulation of p75NTR expression. Neurosci Lett 383, 305-10 (2005).
14. Han, Y., Caday, C. G., Nanda, A., Cavenee, W. K. & Huang, H. J. Tyrphostin AG 1478 preferentially inhibits human glioma cells expressing truncated rather than wild-type epidermal growth factor receptors. Cancer Res 56, 3859-61 (1996).
15. Levitzki, A. & Gazit, A. Tyrosine kinase inhibition: an approach to drug development. Science 267, 1782-8 (1995).
16. Gerecke, K. M., Wyss, J. M. & Carroll, S. L. Neuregulin-1beta induces neurite extension and arborization in cultured hippocampal neurons. Mol Cell Neurosci 27, 379-93 (2004).
17. Gu, Z., Jiang, Q., Fu, A. K., Ip, N. Y. & Yan, Z. Regulation of NMDA receptors by neuregulin signaling in prefrontal cortex. J Neurosci 25, 4974-84 (2005).
18. Sanchez, C., Mendez, C. & Salas, J. A. Indolocarbazole natural products: occurrence, biosynthesis, and biological activity. Nat Prod Rep 23, 1007-45 (2006).
19. Koizumi, S. et al. K-252a: a specific inhibitor of the action of nerve growth factor on PC12 cells. J Neurosci 8, 715-21 (1988).
20. Doherty, P. & Walsh, F. S. K-252a specifically inhibits the survival and morphological differentiation of NGF-dependent neurons in primary cultures of human dorsal root ganglia. Neurosci Lett 96, 1-6 (1989).
21. Perez-Pinera, P. et al. The Trk tyrosine kinase inhibitor K252a regulates growth of lung adenocarcinomas. Mol Cell Biochem (2006).
22. Tamaki, K. et al. Efficient syntheses of novel C2′-alkylated (+/−)-K252a analogues. Org Lett 3, 1689-92 (2001).
23. Roux, P. P. et al. K252a and CEP 1347 are neuroprotective compounds that inhibit mixed-lineage kinase-3 and induce activation of Akt and ERK. J Biol Chem 277, 49473-80 (2002).
24. Maroney, A. C., Sanders, C., Neff, N. T. & Dionne, C. A. K-252b potentiation of neurotrophin-3 is trkA specific in cells lacking p75NTR. J Neurochem 68, 88-94 (1997).
25. Yang, X. et al. Neuregulin-induced expression of the acetylcholine receptor requires endocytosis of ErbB receptors. Mol Cell Neurosci. 28(2), 335-46 (2005).

Other aspects of the invention will be clear to the skilled artisan and need not be repeated here. Each reference cited herein is incorporated by reference in its entirety for the relevant teaching contained therein.

The terms and expressions that have been employed are used as terms of description and not of limitation, and there is no intention in the use of such terms and expressions of excluding any equivalents of the features shown and described or portions thereof, it being recognized that various modifications are possible within the scope of the invention.

We claim:

1.-40. (canceled)

41. A method for treating a subject having or suspected of having a psychotic or cognitive disorder comprising:
administering to a subject in need of such treatment an effective amount of an aminoquinazoline compound as a treatment for the psychotic or cognitive disorder.

42. The method of claim 41, wherein the psychotic or cognitive disorder is a brief psychotic disorder, a delusional disorder, a schizoaffective disorder, schizophrenia, a schizophreniform disorder, a substance-induced psychotic disorder, a psychotic disorder due to a medical condition, paraphrenia, bipolar disorder, psychosis associated with Parkinson's disease, Huntington's disease, manic-depressive psychosis, major depressive disorder with psychotic features, or a shared psychotic disorder.

43. The method of claim 42, wherein the psychotic or cognitive disorder is schizophrenia.

44. The method of claim 43, wherein the schizophrenia is catatonic schizophrenia, disorganized schizophrenia or paranoid schizophrenia.

45. The method of claim 41, wherein the aminoquinazoline compound is gefitinib (Iressa), erlotinib (Tarceva), a salt thereof, or a solvate thereof.

46. The method of claim 41, wherein the subject is a human.

47. A method for treating a subject having or suspected of having a psychotic or cognitive disorder comprising:
administering to a subject in need of such treatment an effective amount of an indolocarbazole compound as a treatment for the psychotic or cognitive disorder.

48. The method of claim 47, wherein the psychotic or cognitive disorder is a brief psychotic disorder, a delusional disorder, a schizoaffective disorder, schizophrenia, a schizophreniform disorder, a substance-induced psychotic disorder, a psychotic disorder due to a medical condition, paraphrenia, bipolar disorder, psychosis associated with Parkinson's disease, Huntington's disease, manic-depressive psychosis, major depressive disorder with psychotic features, or a shared psychotic disorder.

49. The method of claim 48, wherein the psychotic or cognitive disorder is schizophrenia.

50. The method of claim 49, wherein the schizophrenia is catatonic schizophrenia, disorganized schizophrenia or paranoid schizophrenia.

51. The method of claim 47, wherein the indolocarbazole compound is an indolo[2,3-a]carbazole, a salt thereof, or a solvate thereof.

52. The method of claim 51, wherein the indolo[2,3-a]carbazole is furanosylated.

53. The method of claim 52, wherein the furanosylated indolo[2,3-a]carbazole is K252a, analogs thereof, derivatives thereof, a salt thereof, or a solvate thereof.

54. The method of claim 47, wherein the subject is a human.

55.-64. (canceled)
